(19) United States
(12) Patent Application Publication
(10) Pub. No.: US 2006/0281160 A1
(43) Pub. Date: Dec. 14, 2006

(54) Method of hydrogen production in a bioreactor having a circulation system

(76) Inventors: Harry R. Diz, Erie, PA (US); Mitchell S. Felder, Hermitage, PA (US); Justin Felder, Hermitage, PA (US)

Correspondence Address: ECKERT SEAMANS CHERIN & MELLOTT, 600 GRANT STREET, 44TH FLOOR, PITTSBURGH, PA, 15219, US

(21) Appl. No.: 11/450,031

(22) Filed: Jun. 9, 2006

Related U.S. Application Data
(60) Provisional application No. 60/689,485, filed on Jun. 10, 2005.
(60) Provisional application No. 60/692,650, filed on Jun. 21, 2005.

(51) Int. Cl. C12P 3/00

(52) U.S. Cl. 435/168

(57) ABSTRACT

The present invention provides a method of hydrogen production from microorganisms, wherein a bioreactor provides an environment conducive to the production of hydrogen from hydrogen producing microorganisms and restrictive to the production of methane from methanogens. The environment adjusted to a pH conducive to the growth and metabolism hydrogen producing microorganisms within the bioreactor, wherein the pH of the organic feed material is preferably between about 3.5 and 6.0 pH, and by heating the organic feed material prior to entry into the bioreactor. The method further includes a circulation system to create directional flow within the bioreactor.

### CROSS REFERENCE TO RELATED APPLICATIONS

The present application claims priority to U.S. Provisional Ser. Nos. 60/689,485 entitled Hydrogen Producing Bioreactor With Recycling Means, and 60/692,650 entitled Method of Hydrogen Production.

### FIELD OF THE INVENTION

The present invention relates generally to a method for sustained production of hydrogen and concentrated growth of hydrogen generating microorganisms. More particularly, this invention relates to a method for the growth of hydrogen utilizing a bioreactor conducive to the growth of hydrogen producing microorganisms, wherein the bioreactor further includes a circulation system to aid metabolism of the hydrogen producing microorganisms.

### BACKGROUND OF THE INVENTION

The production of hydrogen is an increasingly common and important procedure in the world today. Production of hydrogen in the U.S. alone currently amounts to about 3 billion cubic feet per year, with output likely to increase. Uses for the produced hydrogen are varied, ranging from uses in welding, in production of hydrochloric acid, and for reduction of metallic ores. An increasingly important use of hydrogen, however, is the use of hydrogen in fuel cells or for combustion. This is directly related to the production of alternative fuels for machinery, such as motor vehicles. Successful use of hydrogen as an alternative fuel can provide substantial benefits to the world at large. This is possible not only because hydrogen is produced without dependence on the location of specific oils or other ground resources, but because burning hydrogen is atmospherically clean. Essentially, no carbon dioxide or greenhouse gasses are produced when burning hydrogen. Thus, production of hydrogen as a fuel source can have great impact on the world at large.

For instance, electrolysis, which generally involves the use of electricity to decompose water into hydrogen and oxygen, is a commonly used process. Significant energy, however, is required to produce the needed electricity to perform the process. Similarly, steam reforming is another expensive method requiring fossil fuels as an energy source. As could be readily understood, the environmental benefits of producing hydrogen are at least partially offset when using a process that uses pollution-causing fuels as an energy source for the production of hydrogen.

Thus, producing hydrogen from biological systems, wherein the energy for the process is substantially provided by naturally occurring bacteria, is an optimal solution. Fermentation of organic matter by hydrogen producing microorganisms, such as Bacillus or Clostridium, is one such method. Nonetheless, hydrogen production relating to the above methods has remained problematic, and the need remains for the ability to optimize yields of hydrogen while minimizing expenditures.

New methods of hydrogen generation are needed. One possible method is to convert waste organic matter into hydrogen gas. Microbiologists have for many years known of organisms which generate hydrogen as a metabolic by-product. Two reviews of this body of knowledge are Kosaric and Lyng (1988) and Nandi and Sengupta (1998). Among the various organisms mentioned, the heterotrophic facultative anaerobes of interest in this study, particularly those in the group known as the enteric bacteria. Within this group are the mixed-acid fermenters, whose most well known member is Escherichia coli. While fermenting glucose, these bacteria split the glucose molecule forming two moles of pyruvate (Equation 1); an acetyl group is stripped from each pyruvate fragment leaving formic acid (Equation 2), which is then cleaved into equal amounts of carbon dioxide and hydrogen as shown in simplified form below (Equation 3).
Glucose→2 Pyruvate (1)
2 Pyruvate+2 Coenzyme A→2 Acetyl-CoA+2HCOOH (2)
2HCOOH→2H₂+2CO₂ (3)

Thus, during this process, one mole of glucose produces two moles of hydrogen gas. Also produced during the process are acetic and lactic acids, and minor amounts of succinic acid and ethanol. Other enteric bacteria (the 2,3 butanediol fermenters) use a different enzyme pathway which causes additional CO₂ generation resulting in a 6:1 ratio of carbon dioxide to hydrogen production (Madigan et al., 1997).

There are many sources of waste organic matter which could serve as a substrate for this microbial process, namely as a provider of pyruvate. One such attractive material would be organic-rich industrial wastewaters, particularly sugar-rich waters, such as fruit and vegetable processing wastes. In additional embodiments, wastewaters rich not only in sugars but also in protein and fats could be used, such as milk product wastes. The most complex potential source of energy for this process would be sewage-related wastes, such as municipal sewage sludge and animal manures.

The creation of a gas product that includes hydrogen can be achieved held in a reactor environment favorable to hydrogen production. Substantial, systematic and useful creation of hydrogen gas from microorganisms, however is problematic. The primary obstacle to sustained production of useful quantities of hydrogen by microorganisms has been the eventual stoppage of hydrogen production, generally coinciding with the appearance of methane. This occurs when methanogenic bacteria invade the reactor environment converting hydrogen to methane, typically under the reaction CO₂+4H₂→CH₄+2H₂O. This process occurs naturally in anaerobic environments such as marshes, swamps, pond sediments, and human intestines.

It is of further importance to increase the number of hydrogen producing microorganisms in a system to the point that fixed colonies of biofilm are existent in the bioreactor. Increasing the number of hydrogen producing microorganisms and biofilm and thereby increasing the overall percentage of hydrogen producing microorganisms is beneficial, particularly in large scale reactors. Therefore, it is important to create a bioreactor environment that is conducive to hydrogen producing microorganism growth and maintenance in addition to hydrogen production.

Thus, there continually remains a need to produce substantial and useful levels of hydrogen in a system that provides an environment conducive to metabolism of organic feed material by hydrogen producing microorganisms.

### SUMMARY OF THE INVENTION

The present invention provides a system for aiding the growth of biofilm in a bioreactor, wherein the biofilm is a hydrogen producing microorganisms containing biofilm, wherein the bioreactor is maintained in an environment conducive to the growth of hydrogen producing microorganisms and restrictive to the growth of undesirable organisms including methanogens and the production of methane.

It is an object of the invention to provide a method combining a bioreactor with a circulation system, wherein the circulation system aids metabolism by hydrogen producing microorganisms by circulating organic feed material in a directional flow.

It is a further object of the invention to provide a method for producing hydrogen from hydrogen producing microorganisms metabolizing an organic feed material, having the steps of heating the organic feed material to substantially kill or deactivate methanogens therein, introducing the organic feed material into a bioreactor, adjusting the pH of the organic feed material in the bioreactor to a pH between about 3.5 to 6.0 pH, and circulating the organic feed material, in the bioreactor with a circulation system to create a directional flow in the bioreactor.

It is a further object of the invention to provide a method wherein the pH is monitored with a pH controller.

It is a further object of the invention to provide a method wherein the pH of the organic material is affected when passing through the circulation system.

It is a further object of the invention to provide a method wherein the circulation system includes a pump.

It is a further object of the invention to provide a method wherein the pump is a centrifugal pump.

It is a further object of the invention to provide a method wherein the directional flow in the bioreactor is an up-flow.

It is a further object of the invention to provide a method wherein the directional flow in the bioreactor is a down-flow.

It is a further object of the invention to provide a method further comprising a container for holding a solution that affects pH, wherein the pH is affected by selectively releasing the solution into the organic feed material.

It is a further object of the invention to provide a method wherein a heater heats the organic feed material to a temperature of about 60 to 100° C.

These and other objects of the present invention will become more readily apparent from the following detailed description and appended claims.

### BRIEF DESCRIPTION OF DRAWINGS

FIG. 1 is a plan view of the hydrogen production system.

FIG. 2 is a side view of one embodiment of the bioreactor.

FIG. 3 is a plan view the bioreactor.

FIG. 4 is a plan view of coated substrates.

FIG. 5 is a top plan view of a system layout in a housing unit.

### DETAILED DESCRIPTION OF PREFERRED EMBODIMENTS

As used herein, the term “microorganisms” include bacteria and substantially microscopic cellular organisms.

As used herein, the term “hydrogen producing microorganisms” includes microorganisms that metabolize an organic substrate in one or a series of reactions that ultimately form hydrogen as one of the end products.

As used herein, the term “methanogens” refers to microorganisms that metabolize hydrogen in one or a series of reactions that produce methane as one of the end products.

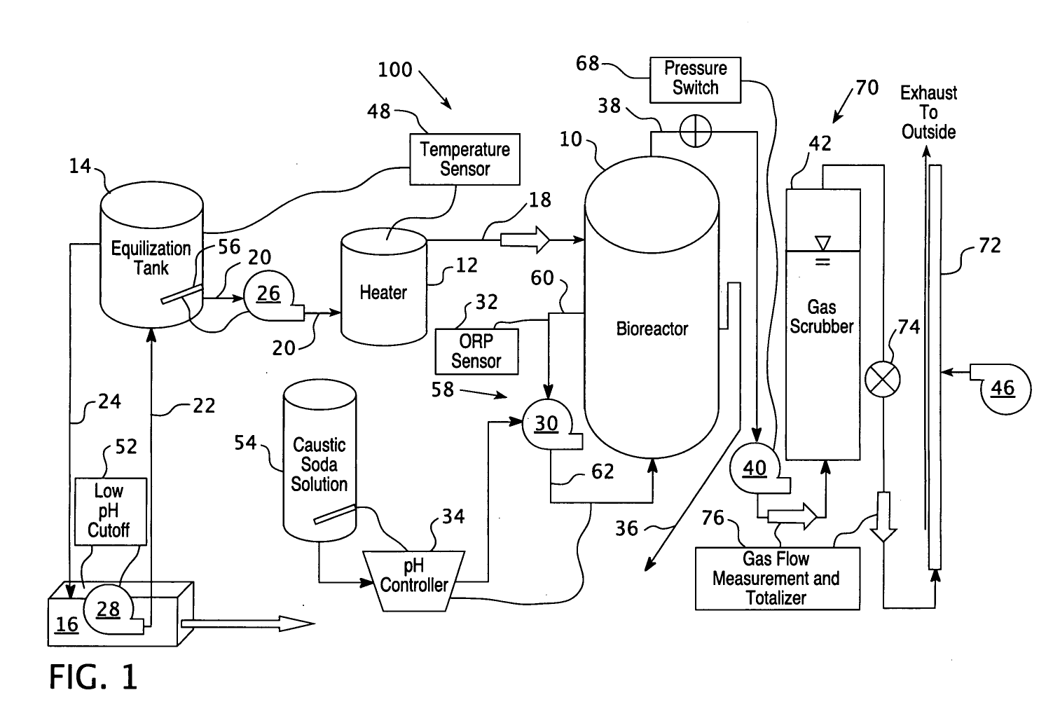

One embodiment of a method for sustained production of hydrogen in accordance with the present invention is shown in FIG. 1, wherein the method uses a system having bioreactor 10, heater 12, equalization tank 14 and reservoir 16. The method enables the production of sustained hydrogen containing gas in bioreactor 10, wherein the produced gas substantially produces a 1:1 ratio of hydrogen to carbon dioxide gas and does not substantially include any methane. The hydrogen containing gas is produced by the metabolism of an organic feed material by hydrogen producing microorganisms. In preferred embodiments, organic feed material is a sugar containing aqueous solution. In further preferred embodiments, the organic feed material is industrial wastewater or effluent product that is produced during routine formation of fruit and/or vegetable juices, such as grape juice. In additional embodiments, wastewaters rich not only in sugars but also in protein and fats could be used, such as milk product wastes. The most complex potential source of energy for this process would be sewage-related wastes, such as municipal sewerage sludge and animal manures. However, any organic feed material organic material is usable.

Hydrogen producing microorganisms metabolize the sugars in the organic feed material under the reactions:
Glucose→2 Pyruvate (1)
2 Pyruvate+2 Coenzyme A→2 Acetyl-CoA+2HCOOH (2)
2HCOOH→2H₂+2CO₂ (3)

During this process, one mole of glucose produces two moles of hydrogen gas and carbon dioxide. In alternate embodiments, other organic feed materials include agricultural residues and other organic wastes such as sewerage and manures. Typical hydrogen producing microorganisms are adept at metabolizing the high sugar organic waste into bacterial waste products. The organic feed material may be further treated by aerating, diluting the organic feed material with water or other dilutants, adding compounds that can control the pH of the organic feed material or other treatment step. For example, the electrolyte contents (Na, K, Cl, Mg, Ca, etc.) of the organic feed material can be adjusted. Further, the organic feed material may be supplemented with phosphorus (NaH₂PO₄) or yeast extract.

Organic feed material provides a plentiful feeding ground for hydrogen producing microorganisms and is naturally infested with these microorganisms. While hydrogen producing microorganisms typically occur naturally in an organic feed material, the organic feed material is preferably further inoculated with hydrogen producing microorganisms in an inoculation step. In further preferred embodiments, the inoculation is an initial, one-time addition to bioreactor 10 at the beginning of the hydrogen production process. The initial inoculation provides enough hydrogen producing microorganisms to create sustained colonies of hydrogen producing microorganisms within the bioreactor. The sustained colonies allow the sustained production of hydrogen. Further inoculations of hydrogen producing microorganisms, however, may be added as desired. The added hydrogen producing microorganisms may include the same types of microorganisms that occur naturally in the organic feed material. In preferred embodiments, the hydrogen producing microorganisms, whether occurring naturally or added in an inoculation step, are preferably microorganisms that thrive in pH levels of about 3.5 to 6.0 and can survive at elevated temperatures. These hydrogen producing microorganisms include, but are not limited to, Clostridium sporogenes, Bacillus licheniformis and Kleibsiella oxytoca. Hydrogen producing microorganisms can be obtained from a microorganisms culture lab or like source. Other hydrogen producing microorganisms or microorganisms known in the art, however, can be used within the spirit of the invention. The inoculation step can occur in bioreactor 10 or elsewhere in the apparatus, for example, circulation system 58.

In one embodiment of the invention, organic feed materiel is first contained in reservoir 16. Reservoir 16 is a container known in the art that can contain an organic feed material. The size, shape, and material of reservoir 16 can vary widely within the spirit of the invention. In one embodiment, reservoir 16 is one or a multiplicity of storage tanks that are adaptable to receive, hold and store the organic feed material when not in use, wherein the one or a multiplicity of storage tanks may be mobile. In preferred embodiments, reservoir 16 is a wastewater well that is adaptable to receive and contain wastewater and/or eluent from all industrial process. In further preferred embodiments, reservoir 16 is adaptable to receive and contain wastewater that is effluent from a juice manufacturing industrial process, such that the effluent held in the reservoir is a Sugar rich juice sludge.

In preferred embodiments of the invention, the method of the invention is used in proximity with an industrial facility. The industrial facility emits waste products, such as organic rich effluent, which is thereafter captured by reservoir 16. By keeping proximity of the method to the industrial facility, the method provides a compact and cost effective method of hydrogen production that conserves energy by using unwanted waste products of an industrial facility to produce hydrogen containing gas.

The organic feed material in reservoir 16 is thereafter conveyed throughout the system, such that the system is preferably a closed system of continuous movement. Conveyance of organic feed material can be achieved by any conveying means known in the art, for example, passages operably related to one or a multiplicity of pumps. The method preferably uses a closed system, such that a few well placed pumps can convey the organic feed material throughout the system, from reservoir 16 to optional equalization tank 14 to heater 12 to bioreactor 10 to outside of bioreactor 10. In preferred embodiments, organic feed material contained in reservoir 16 is conveyed into passage 22 with pump 28. Pump 28 is in operable relation to reservoir 16 such that it aids removal movement of organic feed material 16 into passage 22 at a desired, adjustable flow rate, wherein pump 28 can be any pump known in the art suitable for pumping liquids. In a preferred embodiment, pump 28 is a submersible sump pump.

The method may further include temporary deactivation of conveyance from reservoir 16 to equalization tank 14 or heater 12 if the pH levels of organic feed material in reservoir 16 exceeds a predetermined level. In this embodiment, reservoir 16 furthers include a low pH cutoff device 52, such that exiting movement into passage 22 of the organic feed material is ceased if the pH level of the organic feed material is outside of a desired range. The pH cutoff device 52 is a device known in the alt operably related to reservoir 16 and pump 28. If the monitor detects a pH level of an organic feed material in reservoir 16 out of range, the device ceases operation of pump 28. The pH cut off level in reservoir 16 is typically greater than the preferred pH of bioreactor 10. In preferred embodiments, the pH cutoff level is set between about 7 and 8 pH. The conveyance with pump 28 may resume when the pH level naturally adjusts through the addition of new organic feed material into reservoir 16 or by adjusting the pH through artificial means, such as those of a pH controller. In alternate embodiments, particularly when reservoir 16 is not adapted to receive effluent from an industrial process, the pH cutoff device is not used.

Passage 22 provides further entry access into equalization tank 14 or heater 12. Equalization tank is an optional intermediary container for holding organic feed material between reservoir 16 and heater 12. Equalization tank 14 provides an intermediary container that can help control the flow rates of organic feed material into heater 12 by providing a slower flow rate into passage 20 than the flow rate of organic feed material into the equalization tank through passage 22. An equalization tank is most useful when reservoir 16 received effluent from an industrial facility such that it is difficult to control flow into reservoir 16. The equalization tank can be formed of any material suitable for holding and treating the or organic feed material. In the present invention, equalization tank 14 is constructed of high density polyethylene materials. Other materials include, but are not limited to, metals or plastics. Additionally, the size and shape of equalization tank 14 can vary widely within the spirit of the invention depending on output desired and location limitations.

The method preferably further includes discontinuance of conveyance from equalization tank into heater 12 if the level of organic feed material in equalization tank 14 falls below a predetermined level. Low-level cut-off point device 56 ceases operation of pump 26 if organic feed material contained in equalization tank 14 falls below a predetermined level. This prevents air from being sucked by pump 26 into passage 20, thereby maintaining an anaerobic environment in bioreactor 10. Organic feed material can be removed through passage 20 or through passage 24. Passage 20 provides removal access from equalization tank 14 and entry access into heater 12. Passage 24 provides removal access from equalization tank 14 of organic feed material back to reservoir 16, thereby preventing excessive levels of organic feed material from filling equalization tank 14. Passage 24 provides a removal system for excess organic feed material that exceeds the cut-off point of equalization tank 14. Both passage 20 and passage 24 may further be operably related to pumps to facilitate movement of the organic feed material. In alternate embodiments, equalization tank 14 is not used and organic feed material moves directly from reservoir 16 to heater 12. This is a preferred embodiment when the method is not used in proximate conjunction with industrial facility such that effluent from the industrial facility is directly captured in reservoir 16. If reservoir 16 is one or a multiplicity of storage tanks holding an organic feed material, equalization tank 14 may not be necessary. In these embodiments, passages connecting reservoir 16 and heater 12 are arranged accordingly.

The organic feed material is optionally heated prior to introduction into the bioreactor to deactivate or kill undesirable microorganisms, i.e., methanogens and non-hydrogen producers. The heating can occur anywhere upstream. In one embodiment, the heating is achieved in heater 12, wherein the organic feed material is heated within the heater. Alternatively, organic feed material can be heated at additional or alternate locations in the hydrogen production system. Passage 20 provides entry access to heater 12, wherein heater 12 is any apparatus known in the art that can contain and heat contents held within it. Passage 20 is preferably operably related to pump 26. Pump 26 aids the conveyance of organic feed material from equalization tank 14 or reservoir 16 into heater 12 through passage 20, wherein pump 26 is any pump known in the art suitable for this purpose. In preferred embodiments, pump 26 is an air driven pump for ideal safety reasons, specifically the interest of avoiding creating sparks that could possible ignite hydrogen. However, motorized pumps are also found to be safe and are likewise usable.

To allow hydrogen producing microorganisms within the bioreactor 10 to metabolize the organic teed material and produce hydrogen without subsequent conversion of the hydrogen to methane by methanogens, methanogens contained within the organic feed material are substantially killed or deactivated. In preferred embodiments, the methanogens are substantially killed or deactivated prior to entry into the bioreactor. In further preferred embodiments, methanogens contained within the organic feed material are substantially killed or deactivated by being heated under elevated temperatures in heater 12. Methanogens are substantially killed or deactivated by elevated temperatures. Methanogens are generally deactivated when heated to temperatures of about 60-75° C. for a period of at least 15 minutes. Additionally, methanogens are generally damaged or killed when heated to temperatures above about 90° C. for a period of at least 15 minutes. In contrast, many hydrogen producing microorganisms are resistant to temperatures up to about 110° C. for over three hours. Heater 12 enables heating of the organic feed material to temperature of about 60 to 100° C. in order to substantially deactivate or kill the methanogens while leaving any hydrogen producing microorganisms substantially functional. This effectively pasteurizes or sterilizes the contents of the organic feed material from active methanogens while leaving the hydrogen producing microorganisms intact, thus allowing the produced biogas to include hydrogen without subsequent conversion to methane. Heater 12 can be any receptacle known in the art for holding, receiving and conveying the organic feed material. Similar to the equalization tank 14, heater 12 is preferably formed substantially from metals, acrylics, other plastics or combinations thereof, yet the material can vary widely within the spirit of the invention to include other suitable materials. Similarly, the size and the shape of heater 12 can vary widely within the spirit of the invention depending on output required and location limitations. In preferred embodiments, retention time in heater 12 is at least one hour. Retention time marks the average time any particular part of organic feed material is retained in heater 12.

To maintain the temperatures at desired levels, at least one temperature sensor 48 monitors a temperature indicative of the organic feed material temperature, preferably the temperature levels of equalization tank 14 and/or heater 12. In preferred embodiments, an electronic controller is provided having at least one microprocessor adapted to process signals from one or a plurality of devices providing organic feed material parameter information, wherein the electronic controller is operably related to the at least one actuatable terminal and is arranged to control the operation of and to controllably heat the heating tank and/or any contents therein. The electronic controller is located or coupled to heater 12 or equalization tank 14, or can alternatively be at a third or remote location. In alternate embodiments, the controller for controlling the temperature of heater 12 is not operably related to temperature sensor 48, and temperatures can be adjusted manually in response to temperature readings taken from temperature sensor 48.

Organic feed material is then conveyed from heater 12 to bioreactor 10. Passage 18 connects heater 12 with bioreactor 10. Organic feed material is conveyed into the bioreactor through transport passage 18 at a desired flow rate. When pumps are operating and not shut down by, for example, low pH cut off device 52, the system is preferably a continuous flow system with organic feed material in constant motion between containers such as reservoir 16, heater 12, bioreactor 10, equalization tank 14 if applicable, and so forth. Flow rates in the system can vary depending on retention time desired in any particular container. For example, in preferred embodiments, retention time in bioreactor 10 is between about 6 and 12 hours. To meet this retention time, the flow rate of passage 18 and effluent passage 38 are adjustable as known in the art so that organic feed material, on average, stays in bioreactor 10 for this period of time. In preferred embodiments, pump X also enable conveyance from heater 12 to bioreactor 10 through passage 18. In alternate embodiments, an additional pump can be specifically operably related to passage 18.

The organic feed material is conveyed through passage 18 having a first and second end, wherein passage 18 provides entry access to the bioreactor at a first end of passage 18 and providing removal access to the heater at a second end of passage 18. Any type of passage known in the art can be used, such as a pipe or flexible tube. The transport passage may abut or extend within the bioreactor and/or the heater. Passage 18 can generally provide access into bioreactor 10 at any location along the bioreactor. However, in preferred embodiments, passage 18 provides access at an upper portion of bioreactor 10.

Bioreactor 10 provides an anaerobic environment conducive for hydrogen producing microorganisms to grow, metabolize organic feed material, and produce hydrogen. While the bioreactor is beneficial to the growth of hydrogen producing microorganisms and the corresponding metabolism of organic feed material by the hydrogen producing microorganisms, it is preferably restrictive to the proliferation of methanogens, wherein methanogens are microorganisms that metabolize carbon dioxide and hydrogen to produce methane and water. Methanogens are obviously unwanted as they metabolize hydrogen. If methanogens were to exist in substantial quantities in bioreactor 10, hydrogen produced by the hydrogen producing microorganisms will subsequently be converted to methane, reducing the percentage of hydrogen in the produced gas. Sustained production of hydrogen containing gas is achieved in bioreactor 10 by a number of method steps, including but not limited to providing a supply of organic feed material as a substrate for hydrogen producing microorganisms, controlling the pH of the organic feed material, enabling biofilm growth and other of hydrogen producing microorganisms, and creating directional current in the bioreactor.

Bioreactor 10 can be any receptacle known in the art for carrying an organic feed material. Bioreactor 10 is anaerobic and therefore substantially airtight. Bioreactor 10 itself may contain several openings. However, these openings are covered with substantially airtight coverings or connections, such as passage 18, thereby keeping the environment in bioreactor 10 substantially anaerobic. Generally, the receptacle will be a limiting factor in the amount of material that can be produced. The larger the receptacle, the more hydrogen producing microorganisms containing organic feed material, and, by extension, hydrogen, can be produced. Therefore, the size and shape of the bioreactor can vary widely within the split of the invention depending on output desired and location limitations.

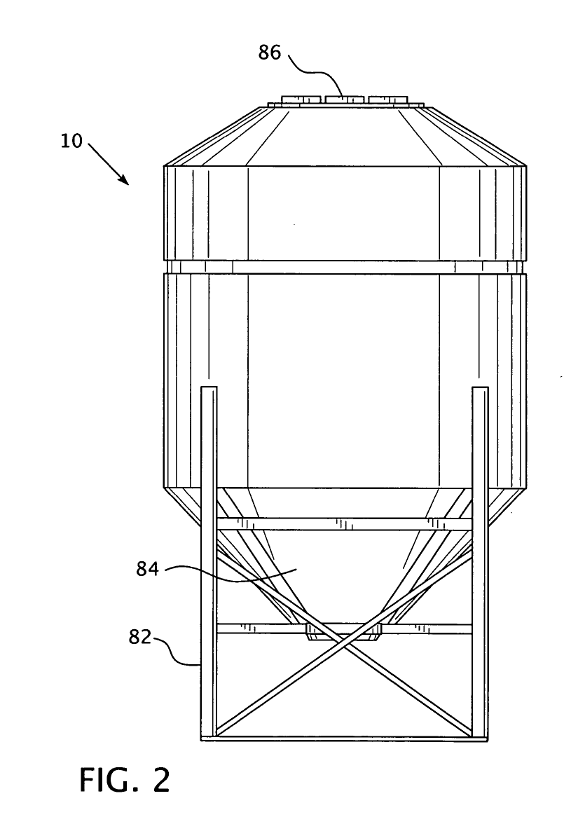

A preferred embodiment of a bioreactor is shown in FIG. 2. Bioreactor 10 can be formed of any material suitable for holding an organic feed material and that can further create all airtight, anaerobic environment. In the present invention, bioreactor 10 is constructed of high density polyethylene materials. Other materials, including but not limited to metals or plastics, can similarly be used. A generally silo-shaped bioreactor 10 has about a 300 gallon capacity with a generally conical bottom 84. Stand 82 is adapted to hold cone bottom 84 and thereby hold bioreactor 10 in an upright position. The bioreactor 10 preferably includes one or a multiplicity of openings that provide a passage for supplying or removing contents from within the bioreactor. The openings may further contain coverings known in the art that cover and uncover the openings as desired. For example, bioreactor 10 preferably includes a central opening covered by lid 86. In alternate embodiments of the invention, the capacity of bioreactor 10 can be readily scaled upward or downward depending on needs or space limitations.

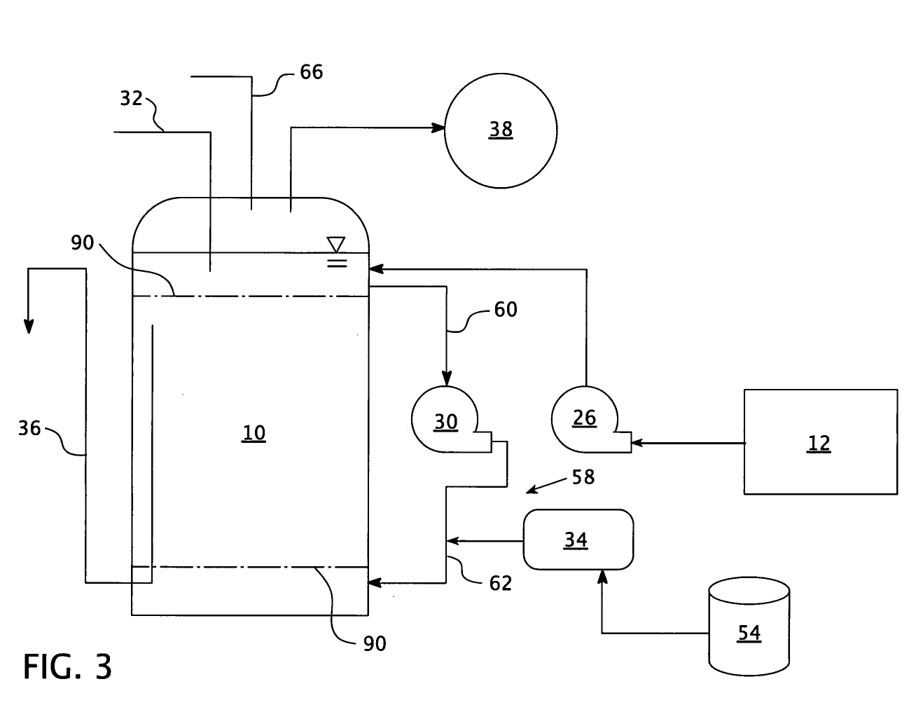

Fresh organic feed material is frequently conveyed into bioreactor 10 to provide new substrate material for the hydrogen producing microorganisms in bioreactor 10. To account for the additional organic feed material and to maintain the organic feed material volume level at a generally constant level, the bioreactor preferably provides a system to remove excess organic feed material, as shown in FIGS. 1 and 3. In the present embodiment, the bioreactor includes effluent passage 36 having an open first and second end that provides a passage from inside bioreactor 10 to outside the bioreactor. The first end of effluent passage 36 may abut bioreactor 10 or extend into the interior of bioreactor 10. If effluent passage 36 extends into the interior of passage 10, the effluent tube preferably extends upwards to generally upper portion of bioreactor 10. When bioreactor 10 is filled with organic feed material, the open first end of the effluent passage allows an excess organic feed material to be received by effluent passage 36. Effluent passage 36 preferably extends from bioreactor 10 into a suitable location for effluent, such as a sewer or effluent container, wherein the excess organic feed material will be deposited through the open second end.

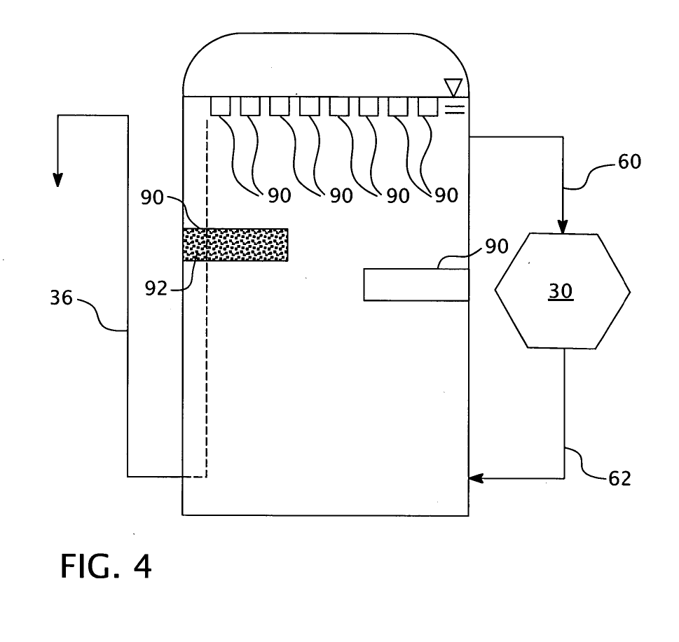

Bioreactor 10 preferably contains one or a multiplicity of substrates 90, as shown in FIG. 4, for providing surface area for attachment and growth of bacterial biofilms. Sizes and shapes of the one or a multiplicity of substrates 90 can vary widely, including but not limited to flat surfaces, pipes, rods, beads, slats, tubes, slides, screens, honeycombs, spheres, object with latticework, or other objects with holes bored through the surface. Numerous substrates can be used, for example, hundreds, as needed. The more successful the biofilm growth on the substrates, the more sustained hydrogen production will be achieved. The fixed nature of the hydrogen producing microorganisms provide the sustain production of hydrogen in the bioreactor.

Substrates 90 preferably are substantially free of interior spaces that potentially fill with gas. In the present embodiment, the bioreactor comprises about numerous pieces of floatable 1″ plastic media to provide surface area for attachment of the bacterial biofilm. In one embodiment, substrates 90 are Flexiring™ Random Packing (Koch-Glitsch.) Some substrates 90 may be retained below the liquid surface by a perforated acrylic plate.

In preferred embodiments, a circulation system 58 is provided in operable relation to bioreactor 10. Circulation system 58 enables circulation of organic feed material contained within bioreactor 10 by removing organic feed material at one location in bioreactor 10 and reintroduces the removed organic feed material at a separate location in bioreactor 10, thereby creating a directional flow in the bioreactor. The directional flow aids the microorganisms within the organic feed material in finding food sources and substrates on which to grown biofilms. As could be readily understood, removing organic feed material from a lower region of bioreactor 10 and reintroducing it at an upper region of bioreactor 10 would create a downward flow in bioreactor 10. Removing organic feed material from an upper region of bioreactor 10 and reintroducing it at a lower region would create an up-flow in bioreactor 10.

In preferred embodiments, as shown in FIG. 1, circulation system 58 is arranged to produce an up-flow of any organic feed material contained in bioreactor 10. Passage 60 provides removal access at a higher point than entry access provided is provided by passage 62. Pump 30 facilitates movement from bioreactor 10 into passage 60, from passage 60 into passage 62, and from passage 62 back into bioreactor 10, creating up-flow movement in bioreactor 10. Pump 30 can be any pump known in the art for pumping organic feed material. In preferred embodiments, pump 30 is an air driven centrifugal pump. Other arrangements can be used, however, while maintaining the spirit of the invention. For example, a pump could be operably related to a single passage that extends from one located of the bioreactor to another.

One or a multiplicity of additional treatment steps can be performed on the organic feed material, either in bioreactor 10 or elsewhere in the system, for the purpose of making the organic feed material more conducive to proliferation of hydrogen producing microorganisms. The one or a multiplicity of treatment steps include, but are to limited to, aerating the organic feed material, diluting the organic feed material with water or other dilutant, controlling the pH of the organic feed material, adjusting electrolyte contents (Na, K, Cl, Mg, Ca, etc.) and adding additional chemical compounds to the organic feed material. Additional chemical compounds added by treatment apparatuses include anti-fungal agents, phosphorous supplements, yeast extract or hydrogen producing microorganisms inoculation. The apparatus performing these treatment steps can be any apparatuses known in the art for incorporating these treatments. For example, in one embodiment, a dilution apparatus is a tank having a passage providing controllable entry access of a dilution, such as water, into bioreactor 10. In some preferred embodiments, the treatment steps are performed in circulation system 58. In other embodiments, treatment steps of the same type may be located at various points in the bioreactor system to provide treatments at desired locations.

Certain hydrogen producing microorganisms proliferate in pH conditions that are not favorable to methanogens, for example, Kleibsiella oxytoca. Keeping organic feed material contained within bioreactor 10 within this favorable pH range is conducive to hydrogen production. Controlling pH in the bioreactor may be performed alternatively or additionally to heating waste material prior to introduction into the bioreactor. In preferred embodiments, pH controller 34 monitors the pH level of contents contained within bioreactor 10. In preferred embodiments, the pH of the organic feed material in bioreactor 10 is maintained at about 3.5 to 6.0 pH, most preferably at about 4.5 to 5.5 pH, as shown in Table 2. In further preferred embodiments, pH controller 34 controllably monitors the pH level of the or organic feed material and adjustably controls the pH of the organic feed material if the organic feed material falls out of or is in danger of falling out of the desired range. As shown in FIG. 1, pH controller 34 monitors the pH level of contents contained in passage 62, such as organic feed material, with a pH sensor (represented as the wavy line connecting pH controller 34 and passage 62.) As could readily be understood, pH controller 34 can be operably related to any additional or alternative location that potentially holds organic feed material, for example, passage 60, 62 or bioreactor 10 as shown in FIG. 3.

If the pH of the organic feed material falls out of a desired range, the pH is preferably adjusted back into the desired range. Control of a pH level provides an environment that enables at least some hydrogen producing microorganisms to function while similarly providing an environment unfavorable to methanogens. This enables microorganisms reactions to create hydrogen without subsequently being overrun by methanogens that convert the hydrogen to methane. Control of pH of the organic feed material in the bioreactor can be achieved by any means known in the art. In one embodiment, a pH controller 34 monitors the pH and can add a pH control solution from container 54 in an automated manner if the pH of the organic feed material moves out of a desired range. In a preferred embodiment, the pH monitor controls the organic feed material's pH through automated addition of a sodium or potassium hydroxide solution. One such apparatus for achieving this is an Etatron DLX pH monitoring device. Preferred ranges of pH for the organic feed material is between about 3.5 and 6.0, with a more preferred range between about 4.0 and 5.5 pH.

The hydrogen producing reactions of hydrogen producing microorganisms metabolizing organic feed material in bioreactor 10 can further be monitored by oxidation-reduction potential (ORP) sensor 32. ORP sensor 32 monitors redox potential of aqueous organic feed material contained within bioreactor 10. Once ORP drops below about −200 mV, gas production commences. Subsequently while operating in a continuous flow mode, the ORP was typically in the range of −300 to −450 mV.

In one embodiment, the organic feed material is a grape juice solution prepared using Welch's Concord Grape Juice™ diluted in chlorine-free tap water at approximately 32 mL of juice per Liter. Alternatively, the solution is aerated previously for 24 hours to substantially remove chlorine. Due to the acidity of the juice, the pH of the organic feed material is typically around 4.0. The constitutional make-up of the grape juice solution is shown in Table 1.

TABLE 1
Composition of concord grape juice. Source:
Welch's Company, personal comm., 2005.

Concentration (unit indicated)

| Constituent | Mean | Range |
|---|---|---|
| Carbohydrates¹ | | 15-18% |
| glucose | 6.2% | 5-8% |
| fructose | 5.5% | 5-8% |
| sucrose | 1.8% | 0.2-2.3% |
| maltose | 1.9% | 0-2.2% |
| sorbitol | 0.1% | 0-0.2% |
| Organic Acids¹ | | 0.5-1.7% |
| Tartaric acid | 0.84% | 0.4-1.35% |
| Malic acid | 0.86% | 0.17-1.54% |
| Citric acid | 0.044% | 0.03-0.12% |
| Minerals¹ | | |
| Calcium | | 17-34 mg/L |
| Iron | | 0.4-0.8 mg/L |
| Magnesium | | 6.3-11.2 mg/L |
| Phosphorous | | 21-28 mg/L |
| Potassium | | 175-260 mg/L |
| Sodium | | 1-5 mg/L |
| Copper | | 0.10-0.15 mg/L |
| Manganese | | 0.04-0.12 mg/L |
| Vitamins¹ | | |
| Vitamin C | | 4 mg/L |
| Thiamine | | 0.06 mg/L |
| Riboflavin | | 0.04 mg/L |
| Niacin | | 0.2 mg/L |
| Vitamin A | | 80 I.U. |
| pH | | 3.0-3.5 |
| Total solids | | 18.5% |

¹additional trace constituents in these categories may be present.

Bioreactor 10 further preferably includes an overflow cut-off switch 66, as shown in FIG. 3, to turn off feed pump 26 if the organic feed material exceeds or falls below a certain level in the bioreactor.

The method further includes capturing hydrogen containing gas produced by the hydrogen producing microorganisms. Capture and cleaning methods can vary widely within the spirit of the invention. In the present embodiment, as shown in FIG. 1, gas is removed from bioreactor 10 through passage 38, wherein passage 38 is any passage known in the art suitable for conveying a gaseous product. Pump 40 is operably related to passage 38 to aid the removal of gas from bioreactor 10 while maintaining a slight negative pressure in the bioreactor. In preferred embodiments, pump 40 is an air driven pump. The gas is conveyed to gas scrubber 42, where hydrogen is separated from carbon dioxide. Other apparatuses for separating hydrogen from carbon dioxide may likewise be used. The volume of collected gas can be measured by water displacement before and after scrubbing with concentrated NaOH. Samples of scrubbed and dried gas may be analyzed for hydrogen and methane by gas chromatography with a thermal conductivity detector (TCD) and/or with a flame ionization detector (FID). Both hydrogen and methane respond in the TCD, but the response to methane is improved in the FID (hydrogen is not detected by an FID, which uses hydrogen as a fuel for the flame).

Exhaust system 70 exhausts gas. Any exhaust system known in the art can be used. In a preferred embodiment, as shown in FIG. 1, exhaust system includes exhaust passage 72, backflow preventing device 74, gas flow measurement and totalizer 76, air blower 46 and exhaust pipe 78.

The organic feed material may be further inoculated in an initial inoculation step with one or a multiplicity of hydrogen producing microorganisms, such as Clostridium sporogenes, Bacillus licheniformis and Kleibsiella oxytoca, while contained in bioreactor 10. These hydrogen producing microorganisms are obtained from a bacterial culture lab or like source. Alternatively, the hydrogen producing microorganisms that occur naturally in the organic feed material can be used without inoculating the organic feed material.

In the present embodiment, the preferred hydrogen producing microorganisms is Kleibsiella oxytoca, a facultative enteric bacterium capable of hydrogen generation. Kleibsiella oxytoca produces a substantially 1:1 ratio of hydrogen to carbon dioxide through organic feed material metabolization, not including impurities. The 1:1 ratio often contains enough hydrogen such that additional cleaning of the produced gas is not necessary. Kleibsiella oxytoca is typically already present in the organic feed material. Alternatively or additionally, the bioreactor may be directly inoculated with Kleibsiella oxytoca. In one embodiment, the inoculum for the bioreactor is a 48 h culture in nutrient broth added to diluted grape juice and the bioreactor was operated until gas production commenced. The bioreactor contents were not stripped of oxygen before or after inoculation.

In further embodiments, the method includes baiting and growing hydrogen producing microorganisms on a carbon-based baiting material provided within bioreactor 10 as shown FIG. 4. In this embodiment, the method further includes a carbon-based baiting material 92, wherein the carbon based material is preferably coated on the one or a multiplicity of substrates 90 within bioreactor 10. The coating baits microorganisms contained in the organic feed material, which then grow thereon.

Carbon based baiting material 92 is preferably a gelatinous matrix having at least one carbon compound. In one embodiment, the gelatinous matrix is alginate or matrix based. In this embodiment, the gelatinous matrix is prepared by placing agar and a carbon compound into distilled water, wherein the agar is a gelatinous mix, and wherein any other gelatinous mix known in the art can be used in place of or in addition to agar within the spirit of the invention.

The carbon compound used with the gelatinous mix to form the gelatinous matrix can vary widely within the spirit of the invention. The carbon source is preferably selected from the group consisting of: glucose, fructose, glycerol, mannitol, asparagines, casein, adonitol, l-arabinose, cellobiose, dextrose, dulcitol, d-galactose, inositol, lactose, levulose, maltose, d-mannose, melibiose, raffinose, rhamnose, sucrose, salicin, d-sorbitol, d-xylose or any combination thereof. Other carbon compounds known in the art, however, can be used within the spirit of the invention.

Generally, the matrix is formed by adding a ratio of three grams of carbon compound and two grams of agar per 100 mL of distilled water. This ratio can be used to form any amount of a mixture up to or down to any scale desired. Once the correct ratio of carbon compound, agar and water are mixed, the mixture is boiled and steam sterilized to form a molten gelatinous matrix. The gelatinous matrix is kept warm within a container such that the mixture remains molten. In one embodiment, the gelatinous matrix is held within a holding container in proximity to substrates 90 until needed to coat the substrates.

The one or a multiplicity of substrates can be any object, shape or material with a hollow or partially hollow interior, wherein the substrate further includes holes that connect the hollow or partially hollow interior to the surface of the substrate. The substrate must also have the ability to withstand heat up to about 110° C. General representative objects and shapes include pipes, rods, beads, slats, tubes, slides, screens, honeycombs, spheres, objects with latticework, or other objects with holes or passages bored through the surface.

In one embodiment, the one or a multiplicity of substrates 90 are generally inserted into the bioreactor through corresponding slots, such that the substrates can be added or removed from the bioreactor without otherwise opening the bioreactor. In alternate embodiments, the substrates are affixed to an interior surface of the bioreactor.

In one embodiment, the one or a multiplicity of substrates are coated by carbon based coating material 92. The substrate can be coated by hand, by machine or by any means known in the art. In one embodiment, the carbon based coating material 92 may be coated directly onto the substrate. In alternative embodiments, however, an adhesive layer may be located between the carbon based coating material 92 and the substrate, the adhesive being any adhesive known in the art for holding carbon based compounds. In a preferred embodiment, the adhesive includes a plurality of gel beads, wherein carbon based coating material 92 is affixed to the gel beads ionically or by affinity.

In additional embodiments, carbon based coating material 92 is conveyed from a container holding carbon based coating material 92 into a hollow or partially hollow interior of the substrate. The gelatinous matrix is conveyed with a pump or other like device into the hollow interior. The carbon based coating material 92 flows from the interior of the substrate to the exterior through the holes, coating the substrate surface. The carbon based coating material 92 on the substrate can be continually replenished at any time by pumping in more gelatinous matrix into the interior of the substrate. The flow of carbon based coating material 92 can be regulated by the conveying device such that the substrate is coated and/or replenished at any speed or rate desired. Further, the entire substrate need not be covered by the carbon based coating material 92, although preferably the majority of the substrate is covered at any moment in time.

The substrate provides an environment for the development and multiplication of microorganisms in the bioreactor. This is advantageous as substrates enable microorganisms to obtain more nutrients and expend less energy than a similar microorganism floating loosely in organic feed material.

The microorganisms, baited by the carbon based coating material, attach themselves to the substrate, thereby forming a slime layer on the substrate generally referred to as a biofilm. The combination of carbon based coating material 92 on the substrate and the environmental conditions favorable to growth in the organic feed material allows the microorganisms to grow, multiply and form biofilms on the substrate.

In order to increase growth and concentration on the substrate coated with a carbon based baiting means for microorganisms, the surface area of the substrate can be increased. Increasing the surface area can be achieved by optimizing the surface area of a single substrate within the bioreactor, adding a multiplicity of substrates within the bioreactor, or a combination of both.

The method may further include coating alginate on the interior of the bioreactor. The thickness and type of alginate coating can vary within the bioreactor. Thus, the bioreactor may have levels of alginate, i.e., areas of different formulations and amounts of alginate in different locations within the bioreactor.

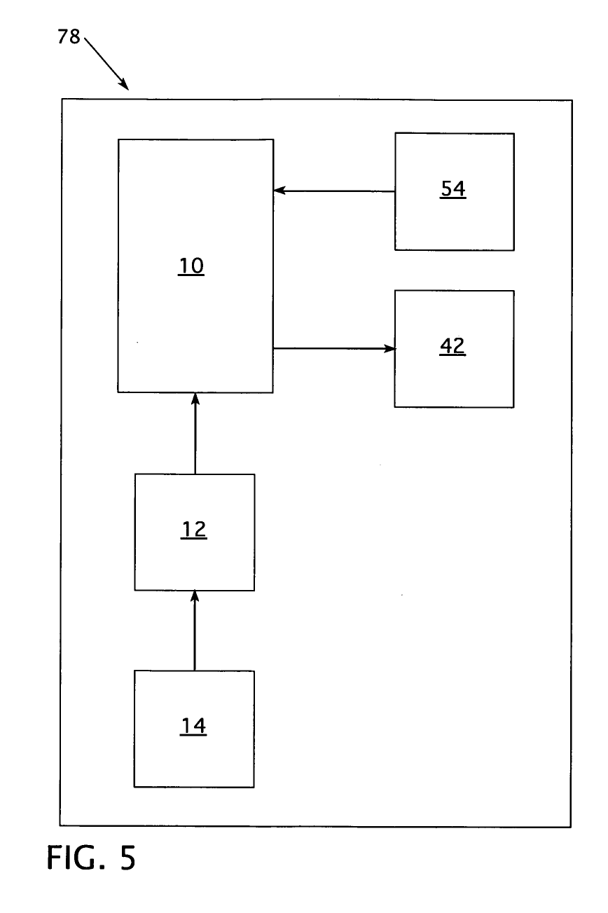

The entire method may be housed in a single housing unit 78 as shown in FIG. 5. The containers and bioreactors will be filled with liquid and thus will be heavy. For example, if a 300 gallon cone-bottom bioreactor is used, the bioreactor can weigh about 3,000 lbs. The stand preferably has four legs, with a 2″ steel plate tying the legs together. If it is assumed that each leg rests on a 2×2 square, then the loading to the floor at those spots would be 190 lbs/sq inch. The inside vertical clearance is preferably at least 84 inches. For safety reasons, the main light switch for the building will be mounted on the outside next to the entry door and the electrical panel will be mounted on the exterior of the building so that all power to the building could be cut without entering. In this further preferred embodiment, the system is preferably proximate to industrial facility.

Hydrogen gas is flammable, but the ignition risk is low, and less than if dealing with gasoline or propane. Hydrogen gas is very light, and will rise and dissipate rapidly. A housing unit is preferably equipped with a vent ridge and eave vents creating natural ventilation. While the LEL (lower explosive limit) for hydrogen is 4%, it is difficult to ignite hydrogen even well above the LEL through electrical switches and motors.

All plumbing connections for the system are water tight, and the gas-side connections are pressure checked. Once the produced gas has been scrubbed of CO2, it will pass through a flow sensor and then be exhausted to the atmosphere through a stand pipe. A blower (as used in boats where gas fumes might be present) will add air to the stand pipe at a rate of more than 500 to 1, thus reducing the hydrogen concentration well below the LEL. As soon as this mixture reaches the top of the pipe, it will be dissipated by the atmosphere.

In case of a leak inside the building, the housing unit preferably includes a hydrogen sensor connected to a relay which will activate an alarm and a ventilation system. The ventilation system is preferably mounted on the outside of the building and will force air through the building and out the roof vents. The hydrogen sensor is preferably set to activate if the hydrogen concentration reaches even 25% of the LEL. The only electrical devices will be a personal computer, low-voltage sensors, electrical outlets and connections, all of which will be mounted on the walls lower than normal. The hydrogen sources will preferably be located high in the room and since hydrogen does not settle.

### EXAMPLE 1

A multiplicity of bioreactors were initially operated at pH 4.0 and a flow rate of 2.5 mL min⁻¹, resulting in a hydraulic retention time (HRT) of about 13 h (0.55 d). This is equivalent to a dilution rate of 1.8 d⁻¹. After one week all six bioreactors were at pH 4.0, the ORP ranged from −300 to −450 mV, total gas production averaged 1.6 L d⁻¹ and hydrogen production averaged 0.8 L d⁻¹. The mean COD of the organic feed material during this period was 4,000 mg L⁻¹ and the mean effluent COD was 2,800 mg L⁻¹, for a reduction of 30%. After one week, the pHs of certain bioreactors were increased by one half unit per day until the six bioreactors were established at different pH levels ranging from 4.0 to 6.5. Over the next three weeks at the new pH settings, samples were collected and analyzed each weekday. It was found that the optimum for gas production in this embodiment was pH 5.0 at 1.48 L hydrogen d⁻¹ (Table 2). This was equivalent to about 0.75 volumetric units of hydrogen per unit of bioreactor volume per day.

TABLE 2
Production of hydrogen in 2-L anaerobic
bioreactors as a function of pH.

| | Total gas | H2 | H2 | H2 per Sugar |
|---|---|---|---|---|
| pH | L/day | L/day | L/g COD | moles/mole |
| 4.0^{a} | 1.61 | 0.82 | 0.23 | 1.81 |
| 4.5^{b} | 2.58 | 1.34 | 0.23 | 1.81 |
| 5.0^{c} | 2.74 | 1.48 | 0.26 | 2.05 |
| 5.5^{d} | 1.66 | 0.92 | 0.24 | 1.89 |
| 6.0^{d} | 2.23 | 1.43 | 0.19 | 1.50 |
| 6.5^{e} | 0.52 | 0.31 | 0.04 | 0.32 |

^{a}mean of 20 data points
^{b}mean of 14 data points
^{c}mean of 11 data points
^{d}mean of 7 data points
^{e}mean of 6 data points

Also shown in Table 2 is the hydrogen production rate per g of COD, which also peaked at pH 5.0 at a value of 0.26 L g⁻¹ COD consumed. To determine the molar production rate, it was assumed that each liter of hydrogen gas contained 0.041 moles, based on the ideal gas law and a temperature of 25° C. Since most of the nutrient value in the grape juice was simple sugars, predominantly glucose and fructose (Table 1 above), it was assumed that the decrease in COD was due to the metabolism of glucose. Based on the theoretical oxygen demand of glucose (1 mole glucose to 6 moles oxygen), one gram of COD is equivalent to 0.9375 g of glucose. Therefore, using those conversions, the molar H₂ production rate as a function of pH ranged from 0.32 to 2.05 moles of H₂ per mole of glucose consumed. As described above, the pathway appropriate to these microorganisms results in two moles of H₂ per mole of glucose, which was achieved at pH 5.0. The complete data set is provided in Tables 3a and 3b.

Samples of biogas were analyzed several times per week from the beginning of the study, initially using a Perkin Elmer Autosystem GC with TCD, and then later with a Perkin Elmer Clarus 500 GC with TCD in series with an FID. Methane was never detected with the TCD, but trace amounts were detected with the FID (as much as about 0.05%).

Over a ten-day period, the organic feed material was mixed with sludge obtained from a methane-producing anaerobic digester at a nearby wastewater treatment plant at a rate of 30 mL of sludge per 20 L of diluted grape juice. There was no observed increase in the concentration of methane during this period. Therefore, it was concluded that the preheating of the feed to 65° C. as described previously was effective in deactivating the microorganisms contained in the sludge. Hydrogen gas production rate was not affected (data not shown).

Using this example, hydrogen gas is generated using a microbial culture over a sustained period of time. The optimal pH for this culture consuming simple sugars from a simulated fruit juice bottling wastewater was found to be 5.0. Under these conditions, using plastic packing material to retain microbial biomass, a hydraulic residence time of about 0.5 days resulted in the generation of about 0.75 volumetric units of hydrogen gas per unit volume of bioreactor per day.

TABLE 3a
Bioreactor Operating Data

| | | | | | | | | | | COD | | | | | | | |
|---|---|---|---|---|---|---|---|---|---|---|---|---|---|---|---|---|---|
| | | | GAS | | Liquid Readings | | | | | | Ef- | Re- | | | Performance | | |
| | | col- | Tot | after | Ef- | | | | | | flu- | mov- | | | Total | | |
| | | lec- | vol- | scrub- | flu- | | Net | | | Feed | ent | al | Load- | Con- | gas | H2 | H2 |
| | Reac- | tion | ume | bing | ent | NaOH | Feed | | | (mg/ | (mg/ | (mg/ | ing | sumed | L/ | L/ | L/g |
| Date | tor | hours | (mL) | (mL) | (mL) | (mL) | (mL) | ORP | pH | L) | L) | L) | (g) | (g) | day | day | COD |
| 17-Nov | C | 5.5 | 360 | 200 | 840 | 120 | 720 | −344 | 4.9 | 4,907 | 2,880 | 2,027 | 3.533 | 1.459 | 1.57 | 0.87 | 0.14 |
| 18-Nov | C | 5 | 370 | 200 | 1120 | 70 | 1050 | −328 | 4.9 | 3,680 | 2,480 | 1,200 | 3.864 | 1.260 | 1.78 | 0.96 | 0.16 |
| 29-Nov | C | 4.25 | 415 | 200 | 920 | 50 | 870 | −403 | 4.9 | 5,013 | 3,093 | 1,920 | 4.362 | 1.670 | 2.34 | 1.13 | 0.12 |
| 17-Nov | E | 5.5 | 490 | 270 | 1210 | 115 | 1095 | −352 | 5.0 | 4,907 | 4,747 | 160 | 5.373 | 0.175 | 2.14 | 1.18 | 1.54 |
| 1-Dec | D | 3.5 | 540 | 250 | 710 | 85 | 625 | −395 | 5.0 | 5,173 | 3,573 | 1,600 | 3.233 | 1.000 | 3.70 | 1.71 | 0.25 |
| 17-Nov | F | 5.5 | 475 | 225 | 1120 | 130 | 990 | −367 | 5.0 | 4,907 | 3,760 | 1,147 | 4.858 | 1.135 | 2.07 | 0.98 | 0.20 |
| 5-Dec | D | 4.5 | 580 | 310 | 710 | 77 | 633 | −423 | 5.0 | 4,267 | 3,573 | 694 | 2.701 | 0.439 | 3.09 | 1.65 | 0.71 |
| 6-Dec | D | 3 | 450 | 240 | 490 | 43 | 447 | −420 | 5.0 | 4,853 | 3,253 | 1,600 | 2.169 | 0.715 | 3.60 | 1.92 | 0.34 |
| 17-Nov | D | 3.5 | 680 | 415 | 580 | 83 | 497 | −326 | 5.0 | 4,907 | 4,213 | 694 | 2.439 | 0.345 | 4.66 | 2.85 | 1.20 |
| 2-Dec | D | 3.75 | 640 | 340 | 830 | 66 | 764 | −412 | 5.0 | 4,587 | 3,787 | 800 | 3.504 | 0.611 | 4.10 | 2.18 | 0.56 |
| 22-Nov | C | 3.75 | 460 | 295 | 800 | 50 | 750 | −349 | 5.0 | 4,107 | 1,280 | 2,827 | 3.080 | 2.120 | 2.94 | 1.89 | 0.14 |
| averages | | 4.34 | 496 | 268 | 848 | 81 | 767 | −374.5 | 5.0 | 4,664 | 3,331 | 1,333 | 3.579 | 1.023 | 2.74 | 1.48 | 0.26 |
| 5-Dec | C | 4.5 | 470 | 250 | 900 | 103 | 797 | −429 | 5.4 | 4,267 | 3,413 | 854 | 3.401 | 0.680 | 2.51 | 1.33 | 0.37 |
| 18-Nov | F | 5 | 90 | 45 | 600 | 55 | 545 | −451 | 5.5 | 3,680 | 3,440 | 240 | 2.006 | 0.131 | 0.43 | 0.22 | 0.34 |
| 21-Nov | D | 4 | 130 | 70 | 830 | 80 | 750 | −454 | 5.5 | 3,493 | 3,360 | 133 | 2.620 | 0.100 | 0.78 | 0.42 | 0.70 |
| 22-Nov | D | 3.75 | 360 | 250 | 766 | 69 | 696 | −461 | 5.5 | 4,107 | 2,880 | 1,227 | 2.858 | 0.854 | 2.30 | 1.60 | 0.29 |
| 29-Nov | D | 4.25 | 100 | 50 | 940 | 100 | 840 | −456 | 5.5 | 5,013 | 3,307 | 1,707 | 4.211 | 1.434 | 0.56 | 0.28 | 0.03 |
| 2-Dec | C | 3.75 | 560 | 290 | 810 | 93 | 717 | −430 | 5.5 | 4,587 | 3,573 | 1,014 | 3.289 | 0.727 | 3.52 | 1.86 | 0.40 |
| 6-Dec | C | 3 | 250 | 130 | 570 | 45 | 525 | −428 | 5.5 | 4,853 | 3,627 | 1,226 | 2.548 | 0.644 | 2.00 | 1.04 | 0.20 |
| averages | | 4.04 | 279 | 155 | 774 | 78 | 696 | −444.1 | 5.5 | 4,286 | 3,371 | 914 | 2.982 | 0.636 | 1.66 | 0.92 | 0.24 |
| 21-Nov | E | 4 | 360 | 250 | 930 | 130 | 800 | −400 | 6.0 | 3,493 | 2,987 | 506 | 2.794 | 0.405 | 2.10 | 1.50 | 0.62 |
| 22-Nov | E | 3.75 | 380 | 280 | 820 | 127 | 693 | −411 | 6.0 | 4,107 | 2,453 | 1,653 | 2.846 | 1.146 | 2.43 | 1.79 | 0.24 |
| 29-Nov | E | 4.25 | 360 | 230 | 870 | 71 | 799 | −467 | 6.0 | 5,013 | 1,973 | 3,040 | 4.006 | 2.429 | 2.03 | 1.30 | 0.09 |
| 1-Dec | E | 3.5 | 420 | 250 | 770 | 127 | 643 | −471 | 6.0 | 5,173 | 2,933 | 2,240 | 3.326 | 1.440 | 2.88 | 1.71 | 0.17 |
| 2-Dec | E | 3.75 | 280 | 170 | 540 | 85 | 455 | −443 | 6.0 | 4,587 | 3,360 | 1,227 | 2.087 | 0.558 | 1.79 | 1.09 | 0.30 |
| 5-Dec | E | 4.5 | 410 | 240 | 930 | 156 | 774 | −487 | 6.0 | 4,267 | 3,253 | 1,014 | 3.303 | 0.785 | 2.19 | 1.28 | 0.31 |
| 6-Dec | E | 3 | 380 | 170 | 660 | 105 | 555 | −490 | 6.0 | 4,853 | 2,293 | 2,560 | 2.693 | 1.421 | 2.24 | 1.36 | 0.12 |
| averages | | 3.82 | 354 | 227 | 789 | 114 | 674 | −453 | 6.0 | 4,499 | 2,750 | 1,749 | 3.033 | 1.179 | 2.23 | 1.43 | 0.19 |
| 29-Nov | F | 4.25 | 90 | 45 | 870 | 150 | 720 | −501 | 6.5 | 5,013 | 1,707 | 3,307 | 3.610 | 2.381 | 0.51 | 0.25 | 0.02 |
| 2-Dec | F | 3.75 | 20 | 0 | 810 | 136 | 674 | −497 | 6.5 | 4,587 | 3,573 | 1,014 | 3.092 | 0.683 | 0.13 | 0.00 | 0.00 |
| 22-Nov | F | 3.75 | 120 | 105 | 790 | 128 | 662 | −477 | 6.5 | 4,107 | 2,240 | 1,867 | 2.719 | 1.236 | 0.77 | 0.67 | 0.08 |
| 5-Dec | F | 4.5 | 10 | 0 | 670 | 121 | 549 | −532 | 6.5 | 4,267 | 2,827 | 1,440 | 2.343 | 0.791 | 0.05 | 0.00 | 0.00 |
| 6-Dec | F | 3 | 60 | 50 | 480 | 90 | 390 | −515 | 6.5 | 4,853 | 2.240 | 2,613 | 1.893 | 1.019 | 0.48 | 0.40 | 0.05 |
| 21-Nov | F | 4 | 200 | 100 | 910 | 150 | 760 | −472 | 6.5 | 3,493 | 2,613 | 880 | 2.655 | 0.669 | 1.20 | 0.60 | 0.15 |
| averages | | 3.88 | 83 | 50 | 755 | 129 | 626 | −499 | 6.5 | 4,387 | 2,533 | 1,853 | 2.745 | 1.160 | 0.52 | 0.31 | 0.04 |

TABLE 3b
Bioreactor Operating Data Continued.

| | | | | | | | | | | COD | | | | | | | |
|---|---|---|---|---|---|---|---|---|---|---|---|---|---|---|---|---|---|
| | | | GAS | | Liquid Readings | | | | | | Ef- | Re- | | | Performance | | |
| | | col- | Total | after | Ef- | | | | | | flu- | mov- | | | Total | | |
| | | lec- | vol- | scrub- | flu- | | Net | | | Feed | ent | al | Load- | Con- | gas | H2 | H2 |
| | Reac- | tion | ume | bing | ent | NaOH | Feed | | | (mg/ | (mg/ | (mg/ | ing | sumed | L/ | L/ | L/g |
| Date | tor | hours | (mL) | (mL) | (mL) | (mL) | (mL) | ORP | pH | L) | L) | L) | (g) | (g) | day | day | COD |
| 14-Nov | A | 5 | 540 | 220 | 780 | 0 | 780 | −408 | 4.0 | 4,480 | 2,293 | 2,187 | 3.494 | 1.706 | 2.59 | 1.06 | 0.13 |
| 14-Nov | B | 5 | 380 | 220 | 840 | 0 | 840 | −413 | 4.1 | 4,480 | 2,453 | 2,027 | 3.763 | 1.702 | 1.82 | 1.06 | 0.13 |
| 14-Nov | C | 5 | 350 | 170 | 870 | 0 | 870 | −318 | 4.1 | 4,480 | 2,293 | 2,187 | 3.898 | 1.902 | 1.68 | 0.82 | 0.09 |
| 14-Nov | D | 5 | 320 | 130 | 920 | 0 | 920 | −372 | 4.1 | 4,480 | 1,920 | 2,560 | 4.122 | 2.355 | 1.54 | 0.62 | 0.06 |
| 14-Nov | E | 5 | 240 | 100 | 920 | 0 | 920 | −324 | 4.3 | 4,480 | 2,773 | 1,707 | 4.122 | 1.570 | 1.15 | 0.48 | 0.06 |
| 14-Nov | F | 5 | 50 | 25 | 810 | 0 | 810 | −329 | 4.0 | 3,307 | 2,080 | 1,227 | 2.679 | 0.994 | 0.24 | 0.12 | 0.03 |
| 15-Nov | A | 5.5 | 450 | 230 | 1120 | 25 | 1095 | −400 | 4.0 | 3,307 | 3,787 | (480) | 3.621 | −0.525 | 1.96 | 1.00 | −0.44 |
| 15-Nov | B | 5.5 | 450 | 235 | 1180 | 35 | 1145 | −384 | 4.0 | 3,307 | 3,253 | 54 | 3.787 | 0.061 | 1.96 | 1.03 | 3.82 |
| 15-Nov | C | 5.5 | 250 | 130 | 640 | 0 | 640 | −278 | 4.0 | 3,307 | 3,520 | (213) | 2.116 | −0.136 | 1.09 | 0.57 | −0.95 |
| 15-Nov | E | 5.5 | 455 | 225 | 1160 | 0 | 1160 | −435 | 4.0 | 3,307 | 3,467 | (160) | 3.836 | −0.185 | 1.99 | 0.98 | −1.21 |
| 15-Nov | F | 5.5 | 430 | 235 | 1160 | 0 | 1160 | −312 | 4.0 | 3,307 | 3,413 | (106) | 3.836 | −0.123 | 1.88 | 1.03 | −1.91 |
| 16-Nov | A | 5 | 380 | 190 | 1020 | 27 | 993 | −414 | 4.0 | 4,693 | 3,627 | 1,066 | 4.660 | 1.059 | 1.82 | 0.91 | 0.18 |
| 5-Dec | A | 4.5 | 200 | 110 | 500 | 35 | 465 | −439 | 4.0 | 4,267 | 4,160 | 107 | 1.984 | 0.050 | 1.07 | 0.59 | 2.21 |
| 18-Nov | A | 5 | 360 | 190 | 200 | 0 | 200 | −423 | 4.0 | 3,680 | 5,227 | (1,547) | 0.736 | −0.309 | 1.73 | 0.91 | −0.61 |
| 21-Nov | A | 4 | 320 | 170 | 800 | 40 | 760 | −429 | 4.0 | 3,493 | 3,680 | (187) | 2.656 | −0.142 | 1.92 | 1.02 | −1.20 |
| 22-Nov | A | 3.75 | 285 | 190 | 725 | 21 | 704 | −432 | 4.0 | 4,107 | 2,293 | 1,813 | 2.891 | 1.277 | 1.82 | 1.22 | 0.15 |
| 29-Nov | A | 4.25 | 310 | 155 | 750 | 24 | 726 | −439 | 4.0 | 5,013 | 3,520 | 1,493 | 3.640 | 1.084 | 1.75 | 0.88 | 0.14 |
| 2-Dec | A | 3.75 | 250 | 120 | 660 | 26 | 634 | −438 | 4.0 | 4,587 | 3,893 | 694 | 2.908 | 0.440 | 1.60 | 0.77 | 0.27 |
| 6-Dec | A | 3 | 150 | 75 | 540 | 0 | 540 | −441 | 4.0 | 4,853 | 3,093 | 1,760 | 2.621 | 0.950 | 1.20 | 0.60 | 0.08 |
| 17-Nov | A | 5.5 | 330 | 160 | 1010 | 30 | 980 | −414 | 4.0 | 4,907 | 3,520 | 1,387 | 4.809 | 1.359 | 1.31 | 0.70 | 0.12 |
| averages | | 4.81 | 324 | 164 | 830 | 13 | 817 | −392 | 4.0 | 4,092 | 3,213 | 879 | 3.344 | 0.718 | 1.61 | 0.82 | 0.23 |
| 16-Nov | B | 5 | 400 | 200 | 1125 | 45 | 1080 | −397 | 4.5 | 4,693 | 3,520 | 1,173 | 5.068 | 1.267 | 1.92 | 0.96 | 0.16 |
| 16-Nov | D | 5 | 400 | 165 | 960 | 60 | 900 | −360 | 4.5 | 4,693 | 3,573 | 1,120 | 4.224 | 1.008 | 1.92 | 0.79 | 0.16 |
| 16-Nov | E | 5 | 490 | 240 | 1100 | 72 | 1028 | −324 | 4.5 | 4,693 | 3,413 | 1,280 | 4.824 | 1.315 | 2.35 | 1.15 | 0.18 |
| 1-Dec | B | 3.5 | 500 | 260 | 570 | 45 | 525 | −415 | 4.5 | 5,173 | 3,680 | 1,493 | 2.716 | 0.784 | 3.43 | 1.78 | 0.33 |
| 6-Dec | B | 3 | 470 | 240 | 650 | 40 | 610 | −411 | 4.5 | 4,853 | 3,360 | 1,493 | 2.960 | 0.911 | 3.76 | 1.92 | 0.26 |
| 21-Nov | B | 4 | 560 | 300 | 930 | 50 | 880 | −397 | 4.5 | 3,493 | 3,147 | 346 | 3.074 | 0.305 | 3.36 | 1.80 | 0.98 |
| 2-Dec | B | 3.75 | 640 | 320 | 830 | 50 | 780 | −407 | 4.5 | 4,587 | 3,413 | 1,174 | 3.578 | 0.915 | 4.10 | 2.05 | 0.35 |
| 17-Nov | B | 5.5 | 450 | 220 | 1165 | 50 | 1115 | −406 | 4.5 | 4,907 | 2,933 | 1,974 | 5.471 | 2.201 | 1.96 | 0.96 | 0.10 |
| 18-Nov | B | 5 | 390 | 220 | 860 | 42 | 818 | −406 | 4.5 | 3,680 | 2,960 | 720 | 3.010 | 0.589 | 1.87 | 1.06 | 0.37 |
| 22-Nov | B | 3.75 | 585 | 395 | 835 | 50 | 785 | −397 | 4.5 | 4,107 | 2,720 | 1,387 | 3.224 | 1.089 | 3.74 | 2.53 | 0.36 |
| 29-Nov | B | 4.25 | 620 | 320 | 920 | 42 | 878 | −410 | 4.5 | 5,013 | 3,307 | 1,707 | 4.402 | 1.498 | 3.50 | 1.81 | 0.21 |
| 5-Dec | B | 4.5 | 390 | 190 | 750 | 37 | 713 | −417 | 4.5 | 4,267 | 3,840 | 427 | 3.042 | 0.304 | 2.08 | 1.01 | 0.62 |
| 16-Nov | F | 5 | 400 | 200 | 1082 | 93 | 989 | −324 | 4.5 | 4,693 | 3,093 | 1,600 | 4.641 | 1.582 | 1.92 | 0.96 | 0.13 |
| 16-Nov | C | 5 | 400 | 200 | 950 | 74 | 876 | −325 | 4.6 | 4,693 | 2,933 | 1,760 | 4.111 | 1.541 | 1.92 | 0.96 | 0.13 |
| averages | | 4.45 | 478 | 248 | 909 | 54 | 856 | −385 | 4.5 | 4,539 | 3,278 | 1,261 | 3.883 | 1.079 | 2.58 | 1.34 | 0.23 |

Whereas particular embodiments of this invention have been described above for purposes of illustration, it will be evident to those skilled in the art that numerous variations of the details of the present invention may be made without departing from the invention as defined in the appended claims.

### SELECTED CITATIONS AND BIBLIOGRAPHY

Brosseau, J. D. and J. E. Zajic. 1982a. Continuous Microbial Production of Hydrogen Gas. Int. J. Hydrogen Energy 7(8): 623-628.
Brosseau, J. D. and J. E. Zajic. 1982ba. Hydrogen-gas Production with Citrobacter intermedius and Clostridium pasteurianum, J. Chem. Tech. Biotechnol, 32:496-502.
Iyer, P., M. A. Bruns, H. Zhang, S. Van Ginkel, and B. E. Logan. 2004. Hydrogen gas production in a continuous flow bioreactor using heat-treated soil inocula. Appl. Microbiol. Biotechnol. 89(1): 119-127.
Kalia, V. C., et al. 1994. Fermentation of biowaste to H2 by Bacillus licheniformis. World Journal of Microbiol & Biotechnol. 10:224-227.
Kosaric, N. and R. P. Lyng. 1988. Chapter 5: Microbial Production of Hydrogen. In Biotechnology, Vol. 6B. editors Rehm & Reed. pp 101-137. Weinheim: Vett.
Logan, B. E., S.-E. Oh, I. S. Kim, and S. Van Ginkel. 2002. Biological hydrogen production measured in batch anaerobic respirometers. Environ. Sci. Technol. 36(11):2530-2535.
Logan, B. E. 2004. Biologically extracting energy from wastewater: biohydrogen production and microbial fuel cells. Environ. Sci. Technol., 38(9):160A-167A
Madigan, M. T., J. M. Martinko, and J. Parker. 1997. Brock Biology of Microorganisms, Eighth Edition, Prentice Hall, New Jersey.
Nandi, R. and S. Sengupta. 1998. Microbial Production of Hydrogen: An Overview. Critical Reviews in Microbiology, 24(1):61-84.
Noike et al. 2002. Inhibition of hydrogen fermentation of organic wastes by lactic acid bacteria. International Journal of Hydrogen Energy. 27:1367-1372
Oh. S.-E. S. Van Ginkel. and B. E. Logan. 2003. The relative effectiveness of pH control and heat treatment for enhancing biohydrogen gas production. Environ. Sci. Technol., 37(22):5186-5190.
Prabha et al. 2003. H₂-Producing bacterial communities from a heat-treated soil Inoculum. Appl. Microbiol. Biotechnol. 66:166-173
Wang et al. 2003. Hydrogen Production from Wastewater Sludge Using a Clostridium Strain. J. Env. Sci. Health. Vol. A38(9):1867-1875
Yokoi et al. 2002. Microbial production of hydrogen from starch-manufacturing wastes. Biomass & Bioenergy; Vol. 22 (5):389-396.

What is claimed is:

1. A method for producing hydrogen from hydrogen producing microorganisms metabolizing an organic feed material, comprising the steps of:
heating the organic feed material to substantially kill or deactivate methanogens therein,
introducing the organic feed material into a bioreactor,
adjusting the pH of the organic feed material in the bioreactor to a pH between about 3.5 to 6.0 pH, and
circulating the organic feed material in the bioreactor with a circulation system to create a directional flow in the bioreactor.

2. The method of claim 1, wherein the pH is monitored with a pH controller.

3. The method of claim 1, wherein the pH of the organic feed material is affected when passing through the circulation system.

4. The method of claim 1, wherein the circulation system includes a pump.

5. The method of claim 4, wherein the pump is a centrifugal pump.

6. The method of claim 1, wherein the directional flow in the bioreactor is an up-flow.

7. The method of claim 1, wherein the directional flow in the bioreactor is a down-flow.

8. The method of claim 1, further comprising a container for holding a solution that affects pH, wherein the pH is affected by selectively releasing the solution into the organic feed material.

9. The method of claim 8, wherein the pH is affected by selectively releasing the solution into the organic feed material as the organic feed material passes through the pump.

10. The method of claim 1, wherein the organic feed material is heated to a temperature of about 60 to 100° C.

11. The method of claim 1, wherein the hydrogen producing microorganisms metabolizing the organic feed material has an ORP between about −300 to −450 mV.

12. A method for producing hydrogen from hydrogen producing microorganisms metabolizing an organic feed material, comprising the steps of:
heating the organic feed material to substantially kill or deactivate methanogens therein,
introducing the organic feed material into a bioreactor,
adjusting the pH of the organic feed material in the bioreactor, and
circulating the organic feed material in the bioreactor with a circulation system to create a directional flow in the bioreactor,
wherein the hydrogen producing microorganisms metabolizing the organic feed material has an ORP between about −300 to −450 mV.

13. The method of claim 12, wherein the pH is adjusted to a pH between about 3.5 and 6.0 pH.

14. The method of claim 12, wherein the pH of the organic feed material is affected when passing through the circulation system.

15. The method of claim 12, wherein the circulation system includes a pump.

16. The method of claim 15, wherein the pump is a centrifugal pump.

17. The method of claim 12, wherein the directional flow in the bioreactor is an up-flow.

18. The method of claim 12, wherein the directional flow in the bioreactor is a down-flow.

19. The method of claim 12, further comprising a container for holding a solution that affects pH, wherein the pH is affected by selectively releasing the solution into the organic feed material.

20. The method of claim 19, wherein the pH is affected by selectively releasing the solution into the organic feed material as the organic feed material passes through the pump.
